(19) United States
(12) Patent Application Publication
(10) Pub. No.: US 2009/0099237 A1
(43) Pub. Date: Apr. 16, 2009

(54) Methods of treating inflammatory diseases

(75) Inventors: Dee Marie Aud, Boulder Creek, CA (US); Stanford Lee-Yu Peng, Palo Alto, CA (US); Kyung Wha Song, Cupertino, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 12/287,605

(22) Filed: Oct. 10, 2008

Related U.S. Application Data
(60) Provisional application No. 60/998,325, filed on Oct. 10, 2007.

(51) Int. Cl. A61K 31/4439; A61P 29/00; A61P 19/02; A61P 11/00; A61P 11/06; A61P 37/00

(52) U.S. Cl. 514/338; 514/341; 514/340

(57) ABSTRACT

Methods relating to selective inhibitors of the casein kinase 1 isoforms that are useful for the treatment of inflammatory diseases are presented.

### CROSS REFERENCE TO RELATED INVENTIONS

This application claims the benefit of priority of U.S. Provisional Patent Application Ser. No. 60/998,325 filed Oct. 10, 2007, which is incorporated herein by reference in its entirety.

### FIELD OF THE INVENTION

The claimed invention relates generally to the fields of medicine and medicinal chemistry. More particularly, the invention relates to methods of treating inflammatory diseases by inhibition of casein kinase 1 isoforms.

### BACKGROUND OF THE INVENTION

Inflammation is a normal part of the response to injuries, invasion by pathogens, and may occur without known cause. The inflammatory process can protect an organism by eliminating pathogens or by removing injured tissue and promoting the restoration of new tissue. However, overabundant or persistent inflammation results in the malfunction or the destruction of vital cells and tissues. Dysregulated inflammation is a hallmark of many painful and life threatening diseases and can affect every tissue and organ of the body. Diseases and disorders which have significant inflammatory components are ubiquitous. Skin disorders, bowel disorders, certain degenerative neurological disorders, arthritis, autoimmune diseases and other illnesses afflict many patients. In certain disorders, infectious agents may be directly or indirectly responsible for the entire disease process. In other disorders, an infectious or other agent may in some way facilitate an autoimmune or inflammatory response. For many patients, dietary or environmental factors may trigger an autoimmune or inflammatory response. In many patients genetic factors can play a key role. In the majority of cases, the causative elements have not been defined and many of the key pathophysiological components have not been elucidated. Accordingly, treatment options for the majority of these diseases is suboptimal.

The casein kinase 1 (CK1) family includes at least 7 ubiquitously-expressed mammalian serine/threonine kinases (isoforms α, β, γ1, γ2, γ3, δ and ε) that generally recognize a consensus sequence S/T(P)-X₁₋₂-S/T, and have been suggested to regulate multiple cellular processes including circadian rhythm, cell growth, proliferation, differentiation and apoptosis (Knippschild et al., Cellular Signalling 2005, 17:675-689). The potential role for CK1 isoforms in inflammatory diseases such as arthritis or asthma, remains largely unknown. However, the observations that CK1 inhibits the activity of nuclear factor of activated T cells (NFAT), a known regulator of lymphocyte activation (Lin & Peng, J. Immunol. 2006, 176:4793-4803) and negatively regulates tumor necrosis factor signaling by phosphorylating the p75 TNF receptor (Beyaert et al., J. Biol. Chem. 1995, 270:23293-23299) tend to suggest that activation of CK1 may result in anti-inflammatory effects.

### SUMMARY OF THE INVENTION

The present invention is based on the surprising discovery that selective inhibitors of casein kinase 1α (CK1α), or casein kinase 1α and casein kinase 1δ (CK1α-CK1δ), or casein kinase 1α and casein kinase 1ε (CK1α-CK1ε), or casein kinase 1α, casein kinase 1δ and casein kinase 1ε (CK1α-CK1δ-CK1ε), are effective in the treatment of inflammatory diseases. Although these enzymes have previously been shown to play a role in the treatment of cancer and neurodegenerative diseases and in circadian rhythm regulation, their roles in treating inflammatory diseases have not been known.

Accordingly, one aspect of the present invention is directed to a method of treating an inflammatory disease in a mammalian subject, comprising administering an effective amount of a selective casein kinase 1α (CK1α) inhibitor, a selective casein kinase 1α-casein kinase 1δ (CK1α-CK1δ) inhibitor, a selective casein kinase 1α-casein kinase 1ε (CK1α-CK1ε) inhibitor or a selective casein kinase 1α-casein kinase 1δ-casein kinase 1ε (CK1α-CK1δ-CK1ε inhibitor. Another aspect of the present invention is directed to a method for identifying compounds that treat an inflammatory disease in a mammalian subject, comprising contacting a compound with CK1α and determining whether the compound selectively inhibits CK1α. A further aspect of the present invention is directed to a method for identifying compounds that treat an inflammatory disease in a mammalian subject, comprising contacting a compound with CK1α, CK1δ, and CK1ε and determining whether the compound selectively inhibits CK1α-CK1δ, CK1α-CK1ε or CK1α-CK1δ-CK1ε.

These and other aspects of the present invention will become evident upon reference to the following detailed description and attached figures. In addition, various references are set forth herein which describe in more detail certain procedures or compositions, and are therefore incorporated by reference in their entirety.

### BRIEF DESCRIPTION OF THE DRAWINGS

FIG. 1 shows the chemical structures of D4476, SB431542, IC261, and Compound A.

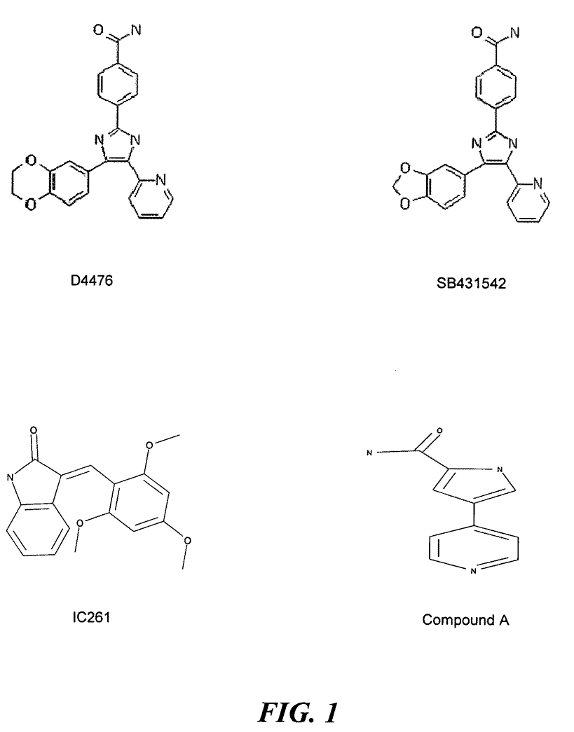

FIG. 2 is a graphical representation of the production of IL-8 in neutrophils following stimulation by inflammatory agents and the dose-dependent inhibition exhibited by SB431542. The dotted line represents unstimulated level of IL-8.

FIG. 3 is a graphical representation of inflammatory neutrophil influx in the air-pouch synovitis model. The inset shows the blockade of inflammatory cytokine accumulation in the same model.

FIG. 4 is a graphical representation of the inhibition of IL-1β or LPS-induced IL-6 production by IC261 and Compound A.

FIG. 5 shows the blockade of IL1-β or LPS-induced IL-6 production by CK1α siRNA (shown in figure as Csnk1α siRNA).

### DETAILED DESCRIPTION OF THE INVENTION

### Definitions

The term “selective casein kinase 1α (CK1α) inhibitor” refers to a substance or compound that inhibits CK1α with an IC₅₀ of less than 10 μM, preferably, less than 3 μM, and does not substantially inhibit other kinases, with the exception of activin-like kinase 5 (Alk5), cell division cycle-like kinase 1 (CLK1), cell division cycle-like kinase 4 (CLK4), serine theronine protein kinase 17A (STK17A), and serine threonine protein kinase 17B (STK17B). The term “selective casein kinase 1α-casein kinase 1δ (CK1α-CK1δ) inhibitor” refers to a substance or compound that inhibits both CK1α and CK1δ with IC₅₀s of less than 10 μM, preferably, less than 3 μM, and does not substantially inhibit other kinases, with the exception of Alk5, CLK1, CLK4, STK17A, and STK17B. The term “selective casein kinase 1α-casein kinase 1ε (CK1α-CK1ε) inhibitor” refers to a substance or compound that inhibits both CK1α and CK1ε with IC₅₀s of less than 10 μM, preferably, less than 3 μM, and does not substantially inhibit other kinases, with the exception of Alk5, CLK1, CLK4, STK17A, and STK17B. The term “selective casein kinase 1α-casein kinase 1δ-casein kinase 1ε (CK1α-CK1δ-CK1ε) inhibitor” refers to a substance or compound that inhibits CK1α, CK1δ and CK1ε with IC₅₀s of less than 10 μM, preferably, less than 3 μM, and does not substantially inhibit other kinases, with the exception of Alk5, CLK1, CLK4, STK17A, and STK17B.

The term “inflammatory diseases” refers to diseases and conditions associated with inflammation which may include but are not limited to: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, and the like, (2) autoimmune diseases, such as arthritis (rheumatoid and psoriatic), osteoarthritis, multiple sclerosis, systemic lupus erythematosus, diabetes mellitus, glomerulonephritis, and the like, (3) graft rejection (including allograft rejection and graft-v-host disease), and (4) other diseases in which undesired inflammatory responses are to be inhibited (e.g., atherosclerosis, myositis, inflammatory CNS disorders such as stroke and closed-head injuries, neurodegenerative diseases, Alzheimer's disease, encephalitis, meningitis, osteoporosis, gout, hepatitis, nephritis, sepsis, sarcoidosis, conjunctivitis, otitis, chronic obstructive pulmonary disease, sinusitis and Bechet's syndrome)

The terms “effective amount” and “therapeutically effective amount” refer to a sufficient amount of the agent to provide the desired biological result. That result can be prevention, reduction and/or alleviation of the signs, symptoms, or causes of a disease, or any other desired alteration of a biological system having or at risk of having such signs, symptoms, or disease. An appropriate “effective” amount in any individual case may be determined by one of ordinary skill in the art using routine experimentation.

The term “treating” or “treatment of” a disease state includes: 1) preventing the disease state, i.e. causing the clinical symptoms of the disease state not to develop in a subject that may be exposed to or predisposed to the disease state, but does not yet experience or display symptoms of the disease state; 2) inhibiting the disease state, i.e., arresting the development of the disease state or its clinical symptoms; 3) or relieving the disease state, i.e., causing temporary or permanent regression of the disease state or its clinical symptoms. The term “disease state” refers to any disease, or pathological condition, symptom, disorder, or indication.

The term “subject” refers to mammals and non-mammals which express CK1α kinase or CK1α kinase and CK1δ kinase and/or CK1ε kinase. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

The terms “pharmaceutically acceptable” and “pharmacologically acceptable” refer to a material that is useful in preparing a pharmaceutical composition that is generally compatible with the other components of the composition, not deleterious to the recipient, and neither biologically nor otherwise undesirable, and is acceptable for veterinary use as well as human pharmaceutical use.

The term, “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentane-propionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo-[2.2.2]oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxy-naphthoic acid, salicylic acid, stearic acid, muconic acid, and the like; or (2) salts formed when an acidic proton present in the parent compound either is replaced by a metal ion, e.g, an alkali metal ion, an alkaline earth ion, or an aluminum ion; or coordinates with an organic base such as ethanolamine, diethanolamine, triethanolamine, tromethamine, N-methylglucamine, and the like.

The term “solvates” refers to solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The term “C₁₋₆alkyl” as used herein whether on its own or as part of a larger group e.g. C₁₋₆alkoxy, refers to a straight or branched chain radical of 1 to 6 carbon atoms, including, but not limited to methyl, ethyl, n-propyl, isopropyl, n-butyl, sec-butyl, isobutyl and tert-butyl.

C₁₋₆haloalkyl groups may contain one or more halo atoms, a particular C₁₋₆haloalkyl group that may be mentioned in CF₃.

The terms “halo” or “halogen” are used interchangeably herein to refer to radicals derived from the elements chlorine, fluorine, iodine and bromine.

The term “C₃₋₇cycloalkyl” as used herein refers to cyclic radicals of 3 to 7 carbons, including but not limited to cyclopropyl, cyclopentyl and cyclohexyl.

The term “aryl” as used herein refers to 5- to 14-membered substituted or unsubstituted aromatic ring(s) or ring systems which may include bi- or tri-cyclic systems, including, but not limited to phenyl and naphthyl.

The term “D4476” refers to the compound, 4-[4-(2,3-Dihydro-benzo[1,4]dioxin-6-yl)-5-pyridin-2-yl-1H-imidazol-2-yl]benzamide. The term “SB431542” refers to the compound, 4-(4-benzo-[1,3]dioxol-5-yl-5-pyridin-2-yl-1H-imidazol-2-yl)benzamide. The term “IC261” refers to the compound, 3-[(2,4,6-trimethoxyphenyl)methylidenyl]-indolin-2-one. The term “Compound A” refers to the compound, 4-Pyridin-4-yl-1H-pyrrole-2-carboxylic acid amide.

General Method

The present invention relates to a method of treating an inflammatory disease in a mammalian subject, comprising administering an effective amount of a selective casein kinase 1α (CK1α) inhibitor, or casein kinase 1α and casein kinase 1δ (CK1α-CK1δ) inhibitor, or casein kinase 1α and casein kinase 1ε (CK1α-CK1ε) inhibitor, or casein kinase 1α, casein kinase 1δ and casein kinase 1ε (CK1α-CK1δ-CK1ε) inhibitor without substantially inhibiting other kinase enzymes. The present invention also relates to a method of for identifying compounds that treat an inflammatory disease in a mammalian subject, comprising contacting compounds with CK1α and determining whether the compound selectively inhibits CK1α or contacting compounds with CK1α, CK1δ, and CK1ε and determining whether the compound selectively inhibits CK1α-CK1δ, CK1α-CK1ε or CK1α-CK1δ-CK1ε.

The selectivity of a given CK1α, CK1α-CK1δ, CK1α-CK1ε or CK1α-CK1δ-CK1ε inhibitor compound can be determined by testing the compound against a panel of known kinases. An example of such a kinase profiling technology is the “KinomeScan” from Ambit Biosciences in which compounds can be screened for their modulatory activities against more than 200 known kinases. Example 2 shows the testing of the compounds of the present invention in a “KinomeScan” screen.

Selective CK1α, CK1α-CK1δ, CK1α-CK1ε or CK1α-CK1δ-CK1ε inhibitors can be tested for their antiinflammatory effects using a variety of in vitro and in vivo tests that are well known in the art. One such in vitro test involves the application of the inhibitor to human neutrophils to observe whether the induction of inflammatory cytokines, such as IL-8, by tumor necrosis factor (TNF) or lipopolysaccharide (LPS) can be reduced. This test is further described in Example 3. An example of an in vivo test, as described in Example 4, is the air pouch model of inflammatory synovitis in mice in which the inhibitors are tested for their blockade of neutrophil influx as well as of inflammatory cytokine accumulation.

Selective inhibitors of CK1α, CK1α-CK1δ, CK1α-CK1ε or CK1α-CK1δ-CK1ε which may find use with the subject methods include synthetic organic molecules, plant extracts and other natural products, and antibodies, antisense molecules and siRNA molecules against CK1α, CK1δ or CK1ε. An example of a selective CK1α-CK1δ-CK1ε inhibitor is D4476, 4-[4-(2,3-Dihydro-benzo[1,4]dioxin-6-yl)-5-pyridin-2-yl-1H-imidazol-2-yl]benzamide, as described by Rena et al. (EMBO Reports 2004, 5:60-65). The compound SB431542, 4-(4-Benzo[1,3]dioxol-5-yl-5-pyridin-2-yl-1H-imidazol-2-yl)benzamide was described in Laping et al. (Molecular Pharmacology 2002, 62:58-64) as an inhibitor against activin-like kinase 5 (Alk5) but has been shown to be a more potent inhibitor of CK1ε activity (Table 1). The synthesis of D4476, SB431542 and compounds of the triarylimidzole series has been described in U.S. Pat. No. 6,465,493, the content of which is incorporated herein by reference. Another selective CK1α-CK1δ-CK1ε inhibitor is IC261, 3-[(2,4,6-trimethoxyphenyl)methylidenyl]-indolin-2-one, as described in Mashhoon et al. (J. Biol. Chem. 2000, 275:20052-20060). Compound A, 4-Pyridin-4-yl-1H-pyrrole-2-carboxylic acid amide, a selective CK1α-CK1δ inhibitor was identified from the Roche Global Chemical Library.

In one embodiment of the present invention, the selective CK1α inhibitor, selective CK1α-CK1δ inhibitor, selective CK1α-CK1ε inhibitor, or selective CK1α-CK1δ-CK1ε inhibitor is a compound of formula (I) or a pharmaceutically acceptable salt or solvate thereof:

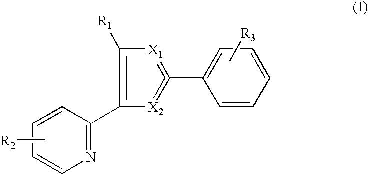

wherein R₁ is naphthyl, anthracenyl, or phenyl optionally substituted with one or more substituents selected from the group consisting of halo, C₁₋₆alkoxy, C₁₋₆alkylthio, C₁₋₆alkyl, —O—(CH₂)ₙ-Ph, —S—(CH₂)ₙ-Ph, cyano, phenyl, and CO₂R, wherein R is hydrogen or C₁₋₆alkyl and n is 0, 1, 2 or 3; or R₁ is phenyl fused with an aromatic or non-aromatic cyclic ring of 5-7 members wherein said cyclic ring optionally contains up to two heteroatoms, independently selected from N, O and S;

R₂ is H, NH(CH₂)ₙ-Ph or NH—C₁₋₆alkyl, wherein n is 0, 1, 2 or 3;

R₃ is CO₂H, CONH₂, CN, NO₂, C₁₋₆alkylthio, —SO₂—C₁₋₆alkyl, C₁₋₆alkoxy, SONH₂, CONHOH, NH₂, CHO, CH₂OH, CH₂NH₂, or CO₂R, wherein R is hydrogen or C₁₋₆alkyl; and

one of X₁ and X₂ is N or CR′, and the other is NR′ or CHR′ wherein R′ is hydrogen, OH, C₁₋₆alkyl, or C₃₋₇cycloalkyl; or when one of X₁ and X₂ is N or CR′ then the other may be S or O.

In another embodiment, inhibitors that can be used in the methods of the present invention include:

4-[4-(4-Fluorophenyl)-5-(2-pyridyl)-1-hydroxy-1H-imidazol-2-yl]benzonitrile;

4-[4-(4-Fluorophenyl)-5-(2-pyridyl)-1H-imidazol-2-yl]benzonitrile;

4-[4-(4-Fluorophenyl)-5-(2-pyridyl)-1H-imidazol-2-yl]benzoic acid;

Methyl4-[4-(4-fluorophenyl)-5-(2-pyridyl)-1H-imidazol-2-yl]benzoate;

Ethyl4-[4-(4-fluorophenyl)-5-(2-pyridyl)-1H-imidazol-2-yl]benzoate;

4-(4-Benzo[1,3]dioxol-5-yl-1-hydroxy-5-pyridin-2-yl-1H-imidazol-2-yl)benzonitrile;

4-(4-Benzo[1,3]dioxol-5-yl-5-pyridin-2-yl-1H-imidazol-2-yl)benzonitrile;

4-(4-Benzo[1,3]dioxol-5-yl-5-pyridin-2-yl-1H-imidazol-2-yl)benzoic acid;

2-[4-Benzo[1,3]dioxol-5-yl-2-(4-nitrophenyl)-1H-imidazol-5-yl]pyridine;

3-(4-Benzo[1,3]dioxol-5-yl-5-pyridin-2-yl-1H-imidazol-2-yl)phenylamine;

4-[4-(4-Fluorophenyl)-2-(4-nitrophenyl)-1H-imidazol-5-yl]pyridine;

4-[4-(4-Fluorophenyl)-5-pyridin-2-yl-1H-imidazol-2-yl)phenylamine;

4-(4-Benzo[1,3]dioxol-5-yl-5-pyridin-2-yl-1H-imidazol-2-yl)phenyl]methanol;

4-(4-Benzo[1,3]dioxol-5-yl-5-pyridin-2-yl-1H-imidazol-2-yl)benzamide;

4-[4-(2,3-Dihydro-benzo[1,4]dioxin-6-yl)-5-pyridin-2-yl-1H-imidazol-2-yl]benzonitrile;

4-[4-(2,3-Dihydro-benzo[1,4]dioxin-6-yl)-5-pyridin-2-yl-1H-imidazol-2-yl]benzamide;

4-[4-(2,3-Dihydro-benzofuran-5-yl)-5-pyridin-2-yl-1H-imidazol-2-yl]benzamide;

3-[4-Benzo[1,3]dioxol-5-yl-5-pyridin-2-yl-1H-imidazol-2-yl)benzonitrile;

4-[4-(2,3-Dihydro-benzofuran-6-yl)-5-pyridin-2-yl-1H-imidazol-2-yl]benzonitrile;

4-[4-(2,3-Dihydro-benzofuran-6-yl)-5-pyridin-2-yl-1H-imidazol-2-yl]benzamide;

3-(4-Benzo[1,3]dioxol-5-yl-5-pyridin-2-yl-1H-imidazol-2-yl)benzoic acid;

4-[4-(4-Methoxyphenyl)-5-(2-pyridyl)-1H-imidazol-2yl]benzonitrile;

4-[4-(2,2-Difluoro-benzo[1,3]dioxol-5-yl)-5-pyridin-2-yl-1H-imidazol-2-yl]benzamide;

4-[4-(2,3-Dihydro-benzo[1,4]dioxin-6-yl)-1-methyl-5-pyridin-2-yl-1H-imidazol-2-yl]benzamide;

4-[5-(2,3-Dihydro-benzo[1,4]dioxin-6-yl)-1-methyl-4-pyridin-2-yl-1H-imidazol-2-yl]benzamide;

4-(5-Benzo[1,3]dioxol-5-yl-4-pyridin-2-yl-oxazol-2-yl)benzonitrile;

4-(5-Benzo[1,3]dioxol-5-yl-4-pyridin-2-yl-oxazol-2-yl)benzamide;

4-(4-Benzo[1,3]dioxol-5-yl-5-pyridin-2-yl-1H-pyrrol-2-yl)benzamide;

and a pharmaceutically acceptable salt or solvate thereof.

In a further embodiment, inhibitors that can be used in the methods of the present invention include 3-[(2,4,6-trimethoxyphenyl)methylidenyl]-indolin-2-one and 4-Pyridin-4-yl-1H-pyrrole-2-carboxylic acid amide.

Administration and Pharmaceutical Composition

The present invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the present invention will be administered in a therapeutically effective amount by any of the accepted modes of administration for agents that serve similar utilities. Suitable dosage ranges are typically about 1-500 mg daily, preferably about 1-100 mg daily, and most preferably about 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this Application, to ascertain a therapeutically effective amount of the compounds of the present invention for a given disease.

In general, compounds of the present invention will be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. The preferred manner of administration is generally oral, using a convenient daily dosage regimen which can be adjusted according to the degree of affliction.

A compound or compounds of the present invention, together with one or more conventional adjuvants, carriers, or diluents, may be placed into the form of pharmaceutical compositions and unit dosages. The pharmaceutical compositions and unit dosage forms may comprise conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the present invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxy-methylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water. The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, nitrogen, nitrous oxide, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinyl-pyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone(1-dodecylazacyclo-heptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa.

### EXAMPLES

The following preparations and examples are given to enable those skilled in the art to more clearly understand and to practice the present invention. They should not be considered as limiting the scope of the invention, but merely as being illustrative and representative thereof.

### Example 1

Kinase Assays:

For in vitro kinase assays, purified recombinant protein CK1α (Invitrogen), CK1δ (Upstate), CK1ε (Invitrogen), or CK1γ1 (Invitrogen) was incubated with 25 μM synthetic peptide (KRRRAL[Ps]VASLPGL) in 30 μL kinase buffer (20 mM MOPS pH 7.2, 25 mM β-glycerol phosphate, 5 mM EGTA, 1 mM sodium orthovanadate, 1 mM DTT, 50 μM ATP, 20 mM MgCl₂, 10 μCi γ-³³P, 0.1% BSA) for the indicated times. A 25 μL aliquot of the reaction mixture was transferred on to p81 phosphocellulose squares (Upstate Biotechnology). The assay squares were washed three times with 0.75% phosphoric acid and once with acetone. Enzyme activity was measured by determining the bound radioactivity by liquid scintillation counting. The IC50 values for several of the tested compounds are shown in Table 1.

TABLE 1

| | D4476 | SB431542 | IC261 | Compound A |
|---|---|---|---|---|
| | IC₅₀ 10 μM | IC₅₀ 10 μM | IC₅₀ 10 μM | IC₅₀ 10 μM |
| CK1α | 1.10 | 1.40 | 6.21 | 1.20 |
| CK1δ | 2.60 | 2.00 | 1.50 | 7.57 |
| CK1ε | 3.50 | 4.10 | 5.97 | 17.3 |
| CK1γ1 | >100 | >100 | >100 | >100 |

### Example 2

Kinase Panel Profiling

To determine the selectivity of D4476, SB431542, IC261 and Compound A for inhibition of kinases, the compounds were tested using the “KinomeScan” kinase profiling technology from Ambit Biosciences (San Diego, Calif.). The results as shown in Tables 2 and 3 demonstrated that at 10 μM concentration, D4476 and SB431542 and IC261 exhibited 100% inhibition against the CK1ε isoform and Compound A showed 94% inhibition at 10 μM concentration.

TABLE 2

| Kinase Gene | % Inhibition | % Inhibition |
|---|---|---|
| (Ambit Symbol) | 10 μM D4476 | 10 μM SB431542 |
| AAK1 | <50 | <50 |
| ABL1 | <50 | <50 |
| ABL2 | <50 | <50 |
| ACK1 | <50 | <50 |
| AKT1 | <50 | <50 |
| AMPK-alpha1 | <50 | <50 |
| AURKA | <50 | <50 |
| AURKC | <50 | <50 |
| BIKE | <50 | <50 |
| BLK | <50 | <50 |
| BMX | <50 | <50 |
| BRAF | <50 | <50 |
| BRAF(V600E) | <50 | <50 |
| BTK | <50 | <50 |
| CAMK1 | <50 | <50 |
| CAMK1D | <50 | <50 |
| CAMK1G | <50 | <50 |
| CAMK2A | <50 | <50 |
| CAMK2B | <50 | <50 |
| CAMK2D | <50 | <50 |
| CAMK2G | <50 | <50 |
| CAMKK1 | <50 | <50 |
| CAMKK2 | <50 | <50 |
| CDK5 | <50 | <50 |
| CLK1 | <50 | <50 |
| CLK2 | <50 | <50 |
| CLK3 | <50 | <50 |
| CLK4 | <50 | <50 |
| CSK | <50 | <50 |
| CSNK1E | 100 | 100 |
| CSNK1G1 | <50 | <50 |
| CSNK1G2 | <50 | <50 |
| CSNK2A1 | <50 | <50 |
| DAPK2 | <50 | <50 |
| DAPK3 | <50 | <50 |
| DMPK | <50 | <50 |
| EGFR | <50 | <50 |
| EPHA2 | <50 | <50 |
| EPHA3 | <50 | <50 |
| EPHA4 | <50 | <50 |
| EPHA5 | <50 | <50 |
| EPHA6 | <50 | <50 |
| EPHA7 | <50 | <50 |
| EPHA8 | <50 | <50 |
| EPHB1 | <50 | <50 |
| EPHB4 | <50 | <50 |
| ERBB2 | <50 | <50 |
| ERBB4 | <50 | <50 |
| ERK2 | <50 | <50 |
| FER | <50 | <50 |
| FES | <50 | <50 |
| FGFR1 | <50 | <50 |
| FGFR2 | <50 | <50 |
| FGFR3 | <50 | <50 |
| FGR | <50 | <50 |
| FLT3 | <50 | <50 |
| FLT4 | <50 | <50 |
| FRK | <50 | <50 |
| FYN | <50 | <50 |
| GAK | <50 | <50 |
| HCK | <50 | <50 |
| IGF1R | <50 | <50 |
| INSR | <50 | <50 |
| ITK | <50 | <50 |
| JAK1(Kin.Dom1) | <50 | <50 |
| JAK2(Kin.Dom2) | <50 | <50 |
| JNK1 | <50 | <50 |
| JNK2 | <50 | <50 |
| JNK3 | <50 | <50 |
| KIT | <50 | <50 |
| LCK | <50 | <50 |
| LIMK1 | <50 | <50 |
| LTK | <50 | <50 |
| LYN | <50 | <50 |
| MAP3K4 | <50 | <50 |
| MAP4K5 | <50 | <50 |
| MARK2 | <50 | <50 |
| MKNK2 | <50 | <50 |
| MYLK2 | <50 | <50 |
| NEK2 | <50 | <50 |
| NEK6 | <50 | <50 |
| NEK9 | <50 | <50 |
| p38-alpha | <50 | <50 |
| p38-beta | <50 | <50 |
| p38-gamma | <50 | <50 |
| PAK1 | <50 | <50 |
| PAK3 | <50 | <50 |
| PAK4 | <50 | <50 |
| PAK6 | <50 | <50 |
| PAK7/PAK5 | <50 | <50 |
| PCTK1 | <50 | <50 |
| PDGFRA | <50 | <50 |
| PDGFRB | <50 | <50 |
| PDPK1 | <50 | <50 |
| PHKG1 | <50 | <50 |
| PHKG2 | <50 | <50 |
| PIM2 | <50 | <50 |
| PKAC-alpha | <50 | <50 |
| PKMYT1 | <50 | <50 |
| PLK4 | <50 | <50 |
| PTK2 | <50 | <50 |
| PTK2B | <50 | <50 |
| PTK6 | <50 | <50 |
| RAF1 | <50 | <50 |
| RIPK2 | <50 | <50 |
| ROS1 | <50 | <50 |
| RPS6KA2 | <50 | <50 |
| RPS6KA3 | <50 | <50 |
| RPS6KA5 | <50 | <50 |
| SLK | <50 | <50 |
| SRC | <50 | <50 |
| STK10 | <50 | <50 |
| STK11 | <50 | <50 |
| STK16 | <50 | <50 |
| STK17A | <50 | <50 |
| STK17B | <50 | <50 |
| STK3 | <50 | <50 |
| STK36 | <50 | <50 |
| STK4 | <50 | <50 |
| SYK | <50 | <50 |
| TIE2 | <50 | <50 |
| TNIK | <50 | <50 |
| TRKA | <50 | <50 |
| TTK | <50 | <50 |
| TXK | <50 | <50 |
| ULK3(mouse) | <50 | <50 |
| VEGFR2 | <50 | <50 |
| YES | <50 | <50 |

TABLE 3

| Kinase Gene | % Binding | % Binding |
|---|---|---|
| (Ambit Symbol) | 10 μM IC261 | 10 μM Cpd A |
| AAK1 | <50 | 69 |
| ABL1 | <50 | <50 |
| ABL2 | <50 | <50 |
| ACK1 | <50 | <50 |
| AKT1 | <50 | <50 |
| AMPK-alpha1 | <50 | <50 |
| AURKA | <50 | <50 |
| AURKC | <50 | <50 |
| BIKE | <50 | <50 |
| BLK | <50 | <50 |
| BMX | <50 | <50 |
| BRAF | <50 | <50 |
| BRAF(V600E) | <50 | <50 |
| BTK | <50 | <50 |
| CAMK1 | <50 | <50 |
| CAMK1D | <50 | <50 |
| CAMK1G | <50 | <50 |
| CAMK2A | <50 | <50 |
| CAMK2B | <50 | <50 |
| CAMK2D | <50 | <50 |
| CAMK2G | <50 | <50 |
| CAMKK1 | <50 | <50 |
| CAMKK2 | <50 | <50 |
| CDK5 | <50 | <50 |
| CLK1 | <50 | 97 |
| CLK2 | <50 | <50 |
| CLK3 | <50 | <50 |
| CLK4 | <50 | 97 |
| CSK | <50 | <50 |
| CSNK1E | 100 | 94 |
| CSNK1G1 | <50 | <50 |
| CSNK1G2 | <50 | 76 |
| CSNK2A1 | <50 | <50 |
| DAPK2 | <50 | <50 |
| DAPK3 | <50 | 74 |
| DMPK | <50 | <50 |
| EGFR | <50 | <50 |
| EPHA2 | <50 | <50 |
| EPHA3 | <50 | <50 |
| EPHA4 | <50 | <50 |
| EPHA5 | <50 | <50 |
| EPHA6 | <50 | <50 |
| EPHA7 | <50 | <50 |
| EPHA8 | <50 | <50 |
| EPHB1 | <50 | <50 |
| EPHB4 | <50 | <50 |
| ERBB2 | <50 | <50 |
| ERBB4 | <50 | <50 |
| ERK2 | <50 | <50 |
| FER | <50 | <50 |
| FES | <50 | <50 |
| FGFR1 | <50 | <50 |
| FGFR2 | <50 | <50 |
| FGFR3 | <50 | <50 |
| FGR | <50 | <50 |
| FLT3 | <50 | <50 |
| FLT4 | <50 | <50 |
| FRK | <50 | <50 |
| FYN | <50 | <50 |
| GAK | <50 | <50 |
| HCK | <50 | <50 |
| IGF1R | <50 | <50 |
| INSR | <50 | <50 |
| ITK | <50 | <50 |
| JAK1(Kin.Dom1) | <50 | <50 |
| JAK2(Kin.Dom2) | <50 | <50 |
| JNK1 | <50 | <50 |
| JNK2 | <50 | <50 |
| JNK3 | <50 | <50 |
| KIT | <50 | <50 |
| LCK | <50 | <50 |
| LIMK1 | <50 | <50 |
| LTK | <50 | <50 |
| LYN | <50 | <50 |
| MAP3K4 | <50 | <50 |
| MAP4K5 | <50 | <50 |
| MARK2 | <50 | <50 |
| MKNK2 | <50 | <50 |
| MYLK2 | <50 | <50 |
| NEK2 | <50 | <50 |
| NEK6 | <50 | <50 |
| NEK9 | <50 | <50 |
| p38-alpha | <50 | <50 |
| p38-beta | <50 | <50 |
| p38-gamma | <50 | <50 |
| PAK1 | <50 | <50 |
| PAK3 | <50 | <50 |
| PAK4 | <50 | <50 |
| PAK6 | <50 | <50 |
| PAK7/PAK5 | <50 | <50 |
| PCTK1 | <50 | <50 |
| PDGFRA | <50 | <50 |
| PDGFRB | <50 | <50 |
| PDPK1 | <50 | <50 |
| PHKG1 | <50 | <50 |
| PHKG2 | <50 | <50 |
| PIM2 | <50 | <50 |
| PKAC-alpha | <50 | <50 |
| PKMYT1 | <50 | <50 |
| PLK4 | <50 | <50 |
| PTK2 | <50 | <50 |
| PTK2B | <50 | <50 |
| PTK6 | <50 | <50 |
| RAF1 | <50 | <50 |
| RIPK2 | <50 | <50 |
| ROS1 | <50 | <50 |
| RPS6KA2 | <50 | <50 |
| RPS6KA3 | <50 | <50 |
| RPS6KA5 | <50 | <50 |
| SLK | <50 | <50 |
| SRC | <50 | <50 |
| STK10 | <50 | <50 |
| STK11 | <50 | <50 |
| STK16 | <50 | <50 |
| STK17A | <50 | 98 |
| STK17B | <50 | 96 |
| STK3 | <50 | <50 |
| STK36 | <50 | <50 |
| STK4 | <50 | <50 |
| SYK | <50 | <50 |
| TIE2 | <50 | <50 |
| TNIK | <50 | <50 |
| TRKA | <50 | <50 |
| TTK | <50 | <50 |
| TXK | <50 | <50 |
| ULK3(mouse) | <50 | <50 |
| VEGFR2 | <50 | <50 |
| YES | <50 | <50 |

### Example 3

Neutrophil Assays

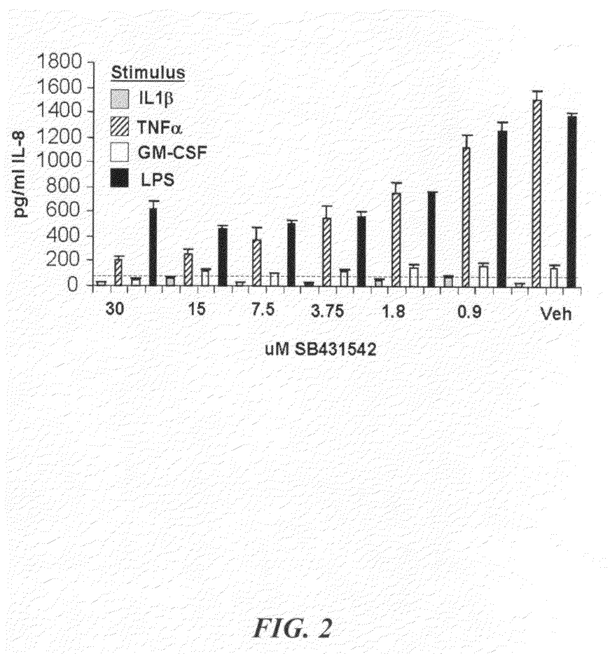

The Histopaque 1077 and 1119 gradient density centrifugation method (Sigma-Aldrich) was used to isolate granulocytes from human blood. The granulocytes were recovered from the 1077/1119 interphase, washed twice with PBS. The red blood cells were lysed with PureGene red cell lysis buffer (Gentra Biosystems). The cells were washed again and resuspended in growth media (RPMI, 10% FBS, β-mercaptoethanol, Pen/Strep/glutamine and sodium pyruvate) at a density of 1 to 5 million per ml. The cells were incubated with vehicle or compound for 30 minutes in a humidified 5% CO₂ incubator. They were then stimulated with one of the following; 100 nM PMA, 10 ng/ml IL1β, 10 ng/ml TNFa, 10 ng/ml or 100 ng/ml LPS for 4 to 8 hours. The supernatant was analyzed for cytokine production (Luminex human 22-plex assay) and the cells were lysed for RNA production and analysis. FIG. 2 shows the dose-dependent inhibition of IL-8 production in neutrophils by SB431542.

### Example 4

Air Pouch Synovitis Model

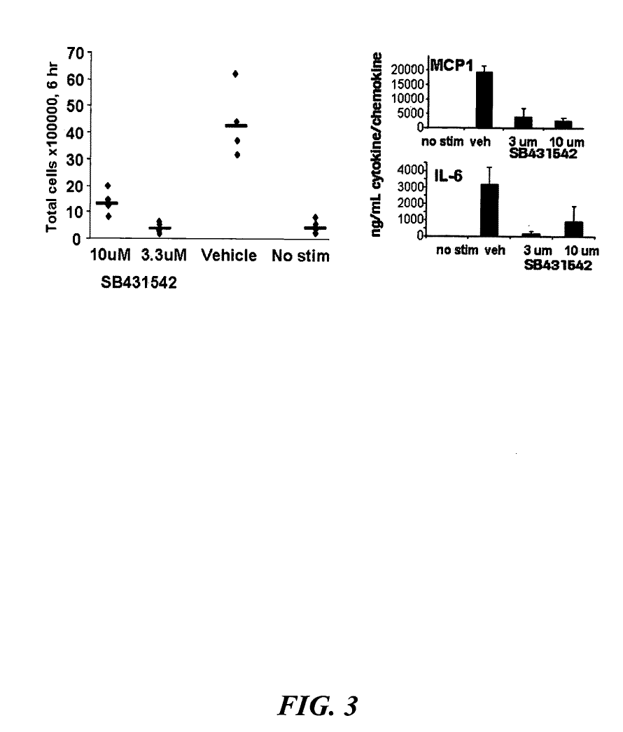

Adult Balbc/J mice were purchased from Charles River Labs and housed and utilized according IACUC protocols and standards. The airpouch was inflated on the dorsal surface of each mouse with 3 ml of air, after light anesthesia with CO₂/O₂ mix. Three days later each pouch was re-inflated with 3 mls of air. After an additional three days, the pouches were injected with vehicle or compound (normally in 0.1% DMSO in PBS) in 1 ml per pouch. After 20 to 30 minutes, each pouch received 1 μg of LPS or control (PBS). The animals were euthanized four to six hours later. The pouches were flushed with 2 ml of PBS, the cells were then collected, counted and normalized for volume recovered. The fluid recovered was analyzed for cytokine production (Luminex Mouse 21-plex assay). The cells were lysed for RNA or protein analysis. FIG. 3 shows that SB431542 inhibited neutrophil influx and the production of the inflammatory mediators, IL-6 and MCP1.

### Example 5

Cytokine-Induced IL-6 Production Assays

Clonetics® primary human umbilical vein endothelial cells (HUVEC; Lonza Walkersville, Inc., Walkersville, Md.) were cultured according to the manufacturer's instructions in EGM™-2 medium (Lonza) supplemented with EGM-2 SingleQuots (containing fetal bovine serum, hydrocortisone, hFGF, VEGF, hFGF-B, R3-IGF-1, ascorbic acid, hEGF, heparin and gentamicin; Lonza). Where indicated, cells were stimulated with 1 ng/mL IL-1β (R&D Systems, Inc., Minneapolis, Minn.) or 50 ng/mL LPS (Sigma-Aldrich, St. Louis, Mo.) for 6 or 24 hours before cell or supernatant harvest.

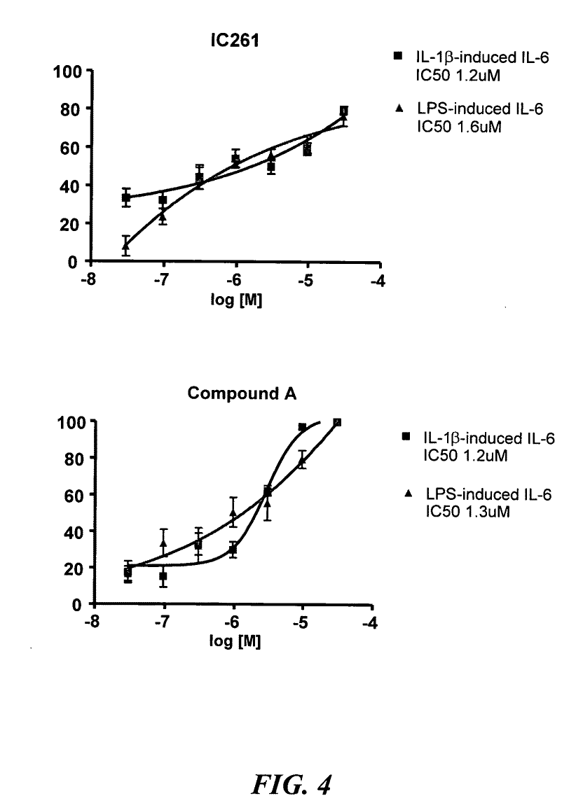

Activity of IC261 and Compound A against cytokine-induced inflammation in vitro was determined in human umbilical vein endothelial cells (HUVECs) upon stimulation with either IL-1β or LPS for 6 hours. IL-6 was assessed in supernatants by ELISA according to the manufacturer's instructions (BD Biosciences), and % inhibition calculated based on maximal IL-6 production in each assay. FIG. 4 shows the results of this study with the IC₅₀ points as calculated by XLFit.

To confirm CK1α as an inflammatory target, siRNA studies were performed. Knockdown studies utilized purified and annealed siRNA duplexes against CK1α, (SMARTpool®; Dharmacon, Lafayette, Colo.), and/or control sequences (scrambled sequences of comparable G/C content and no homology against known targets; Dharmacon). One day before transfection, 1.5×10⁴ HUVECs were cultured in a 96-well plate. On the day of transfection, lipid-RNA complexes (5-100 nM siRNA and 0.3 μL oligofectAMINE in 100 μL OPTI-MEM serum-free medium for a 96-well plate; Invitrogen, Carlsbad, Calif.) were added, and the cells were incubated for 4 hours before media exchange with normal growth medium. Cells were further cultured for 24 hours prior to use.

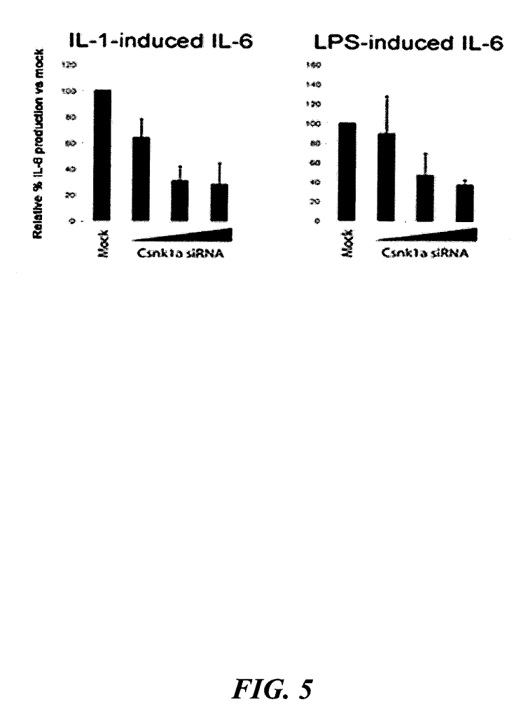

HUVEC cells transfected with CK1α-specific siRNAs were subsequently stimulated by IL-1β or LPS to induce IL-6. Six hours after stimulation, supernatants were assessed for IL-6 levels by ELISA. FIG. 5 shows that increasing concentrations of CK1α siRNAs (10 nM, 50 nM, 100 nM) exhibited greater inhibition of IL-6 production. Control siRNA sequences showed no effect.

What is claimed is:

1. A method of treating an inflammatory disease in a mammalian subject, the method comprising administering an effective amount of a selective casein kinase 1α (CK1α) inhibitor, a selective casein kinase 1α-casein kinase 1δ (CK1α-CK1δ) inhibitor, a selective casein kinase 1α-casein kinase 1ε (CK1α-CK1ε) inhibitor or a selective casein kinase 1α-casein kinase 1δ-casein kinase 1ε (CK1α-CK1δ-CK1ε) inhibitor.

2. The method of claim 1 wherein said selective CK1α inhibitor, selective CK1α-CK1δ inhibitor, selective CK1α-CK1ε inhibitor or selective CK1α-CK1δ-CK1ε inhibitor is a compound of formula (I) or a pharmaceutically acceptable salt or solvate thereof: 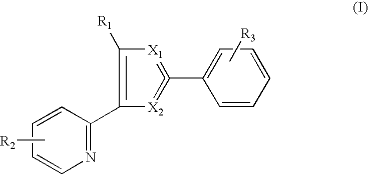
wherein R₁ is naphthyl, anthracenyl, or phenyl optionally substituted with one or more substituents selected from the group consisting of halo, C₁₋₆alkoxy, C₁₋₆alkylthio, C₁₋₆alkyl, —O—(CH₂)ₙ-Ph, —S—(CH₂)ₙ-Ph, cyano, phenyl, and CO₂R, wherein R is hydrogen or C₁₋₆alkyl and n is 0, 1, 2 or 3; or R₁ is phenyl fused with an aromatic or non-aromatic cyclic ring of 5-7 members wherein said cyclic ring optionally contains up to two heteroatoms, independently selected from N, O and S;
R₂ is H, NH(CH₂)ₙ-Ph or NH—C₁₋₆alkyl, wherein n is 0, 1, 2 or 3;
R₃ is CO₂H, CONH₂, CN, NO₂, C₁₋₆alkylthio, —SO₂—C₁₋₄alkyl, C₁₋₆alkoxy, SONH₂, CONHOH, NH₂, CHO, CH₂OH, CH₂NH₂, or CO₂R, wherein R is hydrogen or C₁₋₆alkyl; and
one of X₁ and X₂ is N or CR′, and the other is NR′ or CHR′ wherein R′ is hydrogen, OH, C₁₋₆alkyl, or C₃₋₇cycloalkyl; or when one of X₁ and X₂ is N or CR′ then the other may be S or O.

3. The method of claim 2 wherein said selective CK1α inhibitor, selective CK1α-CK1δ inhibitor, selective CK1α-CK1ε inhibitor or selective CK1α-CK1δ-CK1ε inhibitor is a compound selected from the group consisting of:
4-[4-(4-Fluorophenyl)-5-(2-pyridyl)-1-hydroxy-1H-imidazol-2-yl]benzonitrile;
4-[4-(4-Fluorophenyl)-5-(2-pyridyl)-1H-imidazol-2-yl]benzonitrile;
4-[4-(4-Fluorophenyl)-5-(2-pyridyl)-1H-imidazol-2-yl]benzoic acid;
Methyl4-[4-(4-fluorophenyl)-5-(2-pyridyl)-1H-imidazol-2-yl]benzoate;
Ethyl4-[4-(4-fluorophenyl)-5-(2-pyridyl)-1H-imidazol-2-yl]benzoate;
4-(4-Benzo[1,3]dioxol-5-yl-1-hydroxy-5-pyridin-2-yl-1H-imidazol-2-yl)benzonitrile;
4-(4-Benzo[1,3]dioxol-5-yl-5-pyridin-2-yl-1H-imidazol-2-yl)benzonitrile;
4-(4-Benzo[1,3]dioxol-5-yl-5-pyridin-2-yl-1H-imidazol-2-yl)benzoic acid;
2-[4-Benzo[1,3]dioxol-5-yl-2-(4-nitrophenyl)-1H-imidazol-5-yl]pyridine;
3-(4-Benzo[1,3]dioxol-5-yl-5-pyridin-2-yl-1H-imidazol-2-yl)phenylamine;
4-[4-(4-Fluorophenyl)-2-(4-nitrophenyl)-1H-imidazol-5-yl]pyridine;
4-[4-(4-Fluorophenyl)-5-pyridin-2-yl-1H-imidazol-2-yl)phenylamine;
4-(4-Benzo[1,3]dioxol-5-yl-5-pyridin-2-yl-1H-imidazol-2-yl)phenyl]methanol;
4-(4-Benzo[1,3]dioxol-5-yl-5-pyridin-2-yl-1H-imidazol-2-yl)benzamide;
4-[4-(2,3-Dihydro-benzo[1,4]dioxin-6-yl)-5-pyridin-2-yl-1H-imidazol-2-yl]-benzonitrile;
4-[4-(2,3-Dihydro-benzo[1,4]dioxin-6-yl)-5-pyridin-2-yl-1H-imidazol-2-yl]benzamide;
4-[4-(2,3-Dihydro-benzofuran-5-yl)-5-pyridin-2-yl-1H-imidazol-2-yl]benzamide;
3-[4-Benzo[1,3]dioxol-5-yl-5-pyridin-2-yl-1H-imidazol-2-yl)benzonitrile;
4-[4-(2,3-Dihydro-benzofuran-6-yl)-5-pyridin-2-yl-1H-imidazol-2-yl]benzonitrile;
4-[4-(2,3-Dihydro-benzofuran-6-yl)-5-pyridin-2-yl-1H-imidazol-2-yl]benzamide;
3-(4-Benzo[1,3]dioxol-5-yl-5-pyridin-2-yl-1H-imidazol-2-yl)benzoic acid;
4-[4-(4-Methoxyphenyl)-5-(2-pyridyl)-1H-imidazol-2yl]benzonitrile;
4-[4-(2,2-Difluoro-benzo[1,3]dioxol-5-yl)-5-pyridin-2-yl-1H-imidazol-2-yl]benzamide;
4-[4-(2,3-Dihydro-benzo[1,4]dioxin-6-yl)-1-methyl-5-pyridin-2-yl-1H-imidazol-2-yl]benzamide;
4-[5-(2,3-Dihydro-benzo[1,4]dioxin-6-yl)-1-methyl-4-pyridin-2-yl-1H-imidazol-2-yl]benzamide;
4-(5-Benzo[1,3]dioxol-5-yl-4-pyridin-2-yl-oxazol-2-yl)benzonitrile;
4-(5-Benzo[1,3]dioxol-5-yl-4-pyridin-2-yl-oxazol-2-yl)benzamide;
4-(4-Benzo[1,3]dioxol-5-yl-5-pyridin-2-yl-1H-pyrrol-2-yl)benzamide;
and a pharmaceutically acceptable salt or solvate thereof.

4. The method of claim 3 wherein said selective CK1α inhibitor, selective CK1α-CK1δ inhibitor, selective CK1α-CK1ε inhibitor or selective CK1α-CK1δ-CK1ε inhibitor is a compound selected from 4-(4-Benzo[1,3]dioxol-5-yl-5-pyridin-2-yl-1H-imidazol-2-yl)benzamide or 4-[4-(2,3-Dihydro-benzo[1,4]dioxin-6-yl)-5-pyridin-2-yl-1H-imidazol-2-yl]benzamide, and pharmaceutically acceptable salts or solvates thereof.

5. The method of claim 1 wherein said selective CK1α inhibitor, selective CK1α-CK1δ inhibitor, selective CK1α-CK1ε inhibitor or selective CK1α-CK1δ-CK1ε inhibitor is a compound selected from 3-[(2,4,6-trimethoxyphenyl)methylidenyl]-indolin-2-one and 4-Pyridin-4-yl-1H-pyrrole-2-carboxylic acid amide, and pharmaceutically acceptable salts or solvates thereof.

6. The method of claim 1, 2, 3, 4 or 5 wherein said inflammatory disease is selected from the group consisting of osteoarthritis, rheumatoid arthritis, asthma, chronic obstructive pulmonary disease, inflammatory bowel disease, lupus erythematosus, multiple sclerosis and inflammatory CNS disorders.

7. The method of claim 6 wherein said inflammatory disease is rheumatoid arthritis or asthma.

8. A method for identifying compounds that treat an inflammatory disease in a mammalian subject, the method comprising contacting a compound with CK1α and determining whether the compound selectively inhibits CK1α.

9. The method of claim 8 comprising contacting a compound with CK1α, CK1δ and CK1ε and determining whether the compound selectively inhibits CK1α-CK1δ, CK1α-CK1ε or CK1α-CK1δ-CK1ε.

10. The method of claim 8 or 9 wherein said inflammatory disease is chosen from the group consisting of osteoarthritis, rheumatoid arthritis, asthma, chronic obstructive pulmonary disease, inflammatory bowel disease, lupus erythematosus, multiple sclerosis and inflammatory CNS disorders.

11. The method of claim 10 wherein said inflammatory disease is rheumatoid arthritis or asthma.
